(19) United States
(12) Patent Application Publication
(10) Pub. No.: US 2020/0405532 A1
(43) Pub. Date: Dec. 31, 2020

(54) DEVICES, SYSTEMS, AND METHODS FOR THE TREATMENT OF CLOGGED GLANDS OF THE EYE

(71) Applicant: Please Me LLC, Brooklyn, NY (US)

(72) Inventors: Carolyn Sitt, Brooklyn, NY (US); Eddie Sitt, Brooklyn, NY (US)

(73) Assignee: Please Me LLC, Brooklyn, NY (US)

(21) Appl. No.: 16/913,870

(22) Filed: Jun. 26, 2020

Related U.S. Application Data
(60) Provisional application No. 62/866,846, filed on Jun. 26, 2019.

(51) Int. Cl. A61F 7/02

(52) U.S. Cl. CPC A61F 7/02; A61F 9/04

(57) ABSTRACT

A heated eye mask for treating clogged glands of an eye includes a mask body. The mask body includes a first layer of surface material configured to be positioned away from an eye region of and a second layer of surface material configured to contact the eye region, a heating element disposed between the first and second layers of surface material in a therapeutic region of the mask body, and a thermally conductive material in contact with the heating element and disposed between the heating element and the second layer of surface material to evenly distribute heat across the therapeutic region of the mask body. The heated eye mask further includes an electrical cord configured to be coupled with a power source and in communication with the heating element and an adjustable strap to maintain contact of the mask body with the eye region of the user.

### CROSS-REFERENCE TO RELATED PATENT APPLICATIONS

This application claims the benefit of and priority to U.S. Provisional Patent Application No. 62/866,846, filed Jun. 26, 2019, which is hereby incorporated by reference in its entirety.

### BACKGROUND

The present invention relates generally to the field of treatment for clogged glands of the eye, and particularly, to a thermal compress for providing heat to an eye region for treating the clogged glands.

Many people experience dry eye syndrome. One of the causes of dry eyes is that the oil glands of the eyes, known as the Meibomian glands, become clogged. One condition that relates to a blockage or other abnormality of the Meibomian glands is referred to as Meibomian gland dysfunction (MGD). For a person with MGD, the Meibomian glands do not secrete enough oil into the eyes. When tears are inside the eye, they will quickly evaporate unless there is a layer of oil on top. The oil prevents evaporation of tears and also helps lubricate the eyes. Because the tears then evaporate too quickly, MGD is associated with dry eye syndrome. MGD also relates to an eyelid problem called blepharitis which causes inflammation of the eyelids.

A common recommendation from medical professionals for treatment of dry eye syndrome and/or blepharisis is to take a fabric eye mask, wet it, and then heat it in the microwave for 20 seconds before applying to the eye region to unclog the glands. There are two primary issues associated with this method. First, the heat very quickly dissipates and becomes ineffective. Second, the fabric on the mask is not directly focused on the target area, but rather heats up the entire general eye area including the eye lids, eyebrows, upper cheeks, etc.

There is, therefore, a need for a more effective treatment of MGD, dry eye syndrome, blepharitis, and/or any other condition that involves clogged glands in the eye region.

### SUMMARY OF THE INVENTION

One embodiment relates to a heated eye mask for treating clogged glands of an eye. The heated eye mask includes a mask body, which includes a first layer of surface material configured to be positioned away from an eye region of a user and a second layer of surface material configured to contact the eye region of the user, a heating element disposed between the first and second layers of surface material in a therapeutic region of the mask body, and a thermally conductive material in contact with the heating element and disposed between the heating element and the second layer of surface material to evenly distribute heat across the therapeutic region of the mask body. The heated eye mask further includes a power connector configured to be coupled with a power source and in communication with the heating element, and an adjustable strap to maintain contact of the mask body with the eye region of the user.

Another embodiment relates to a system for treating clogged glands of an eye. The system includes a heated eye mask, an electrical cord coupled to the heated eye mask for providing power to the heated eye mask, and a controller provided on the electrical cord for controlling the heated eye mask.

Another embodiment relates to a method for treating clogged glands of an eye. The method includes positioning a heated eye mask on an eye region, setting at least one of a therapy temperature and a therapy time of use using a controller coupled to the heated eye mask, providing power to the heated eye mask by connecting the heated eye mask to a power supply, and maintaining the heated eye mask on the eye region at the set temperature and for a desired amount of time.

### BRIEF DESCRIPTION OF THE DRAWINGS

FIG. 1 depicts the human eye showing the Meibomian glands;

FIG. 2 shows a system for treatment of clogged glands of the eye;

FIG. 3 shows an electric heated eye mask of the system of FIG. 2 having an alternative power source configuration;

FIG. 4 shows an electric heated eye mask for treatment of clogged glands of the eye;

FIG. 5 is an alternative view of the electric heated eye mask shown in FIG. 4;

FIG. 6A is a view of the arrangement of the internal elements of the electric heated eye mask of FIG. 4;

FIG. 6B is an alternative view of the arrangement of the internal elements of the electric heated eye mask of FIG. 4;

FIG. 7 depicts a heat controller for use with an electric heated eye mask;

FIG. 8 is a flowchart depicting a method for treating clogged glands of the eye.

### DETAILED DESCRIPTION

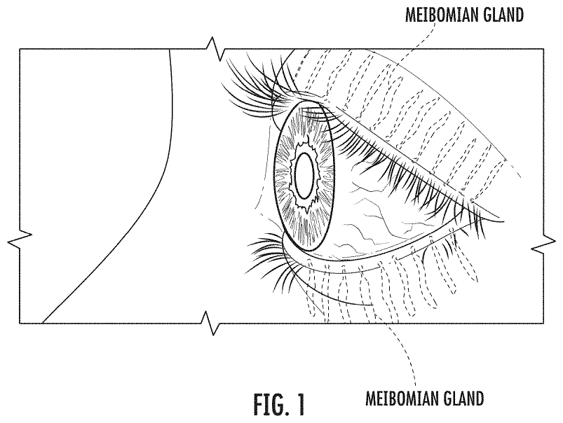

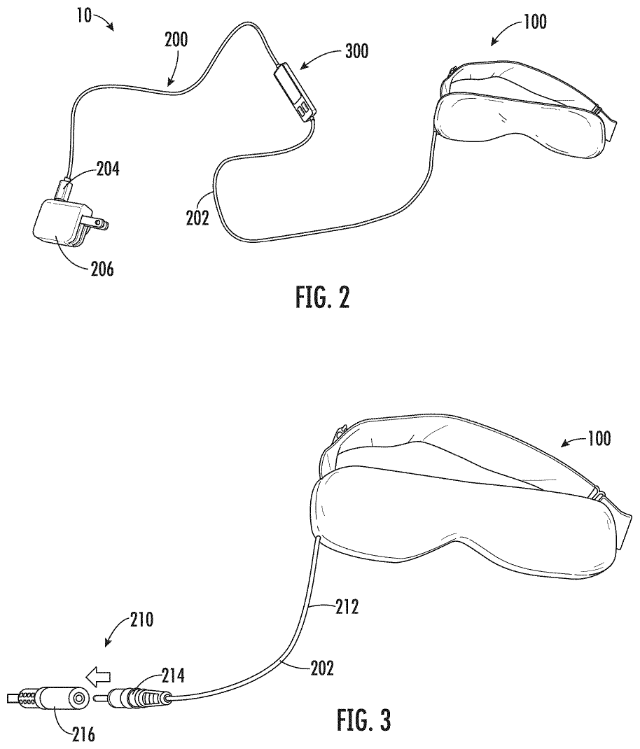

One cause of dry or irritated eyes is blockage of the Meibomian glands of the eyes. The Meibomian glands are depicted in FIG. 1. The Meibomian glands provide oils to the eyes for protection and moisture. FIG. 2 depicts a system 10 for treating clogged glands of an eye, such as the Meibomian glands, by providing heat to melt the oils in the glands, and therefore, unclog the glands.

As shown in FIG. 2, the system 10 includes a heated eye mask 100, a power source 200, and a controller 300. The heated eye mask 100 is configured to be worn by a user by positioning and securing the mask 100 over the user's eye region. The power source 200 provides power to the eye mask 100 for heating the eye mask 100 during a treatment session. The controller 300 is coupled to the power source 200 to control the time of the treatment session and/or the temperature provided by the eye mask 100 during the treatment session.

The power source 200, shown in FIG. 2, is an electrical cord 202 with a USB interface 204 at its distal end. In such embodiments, the USB interface 204 is configured to be coupled to any USB power device such as a 5V adapter 206 for a wall outlet, a battery pack, a personal computer, a USB power hub, etc. In an alternative embodiment, shown in FIG. 3, there is a disconnectable portion 210 along the cord 202. Specifically, a power connector 212 extends from the eye mask 100. The power connector 212 is a lead wire having a socket 214 at its distal end. The socket 214 is configured to receive a DC plug 216 which continues to an interface, such as the USB interface 204, for plugging into a power supply.

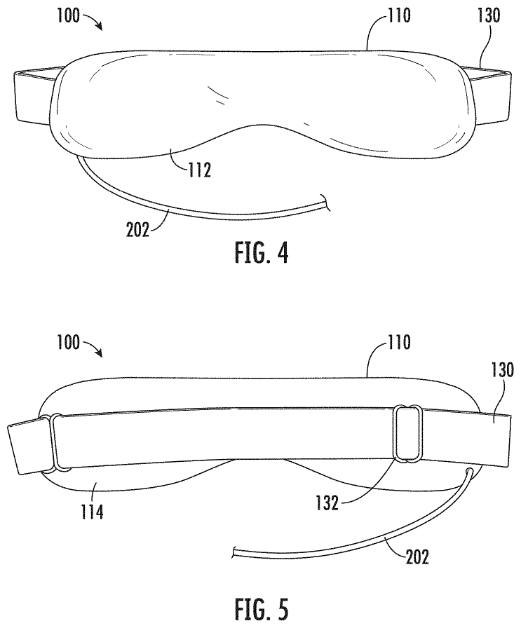

Referring to FIGS. 4-5, the heated eye mask 100 is shown in greater detail. FIG. 4 is a front view of the heated eye mask 100, showing an outside of the mask 100 that would face away from a user. FIG. 5 is a rear view of the heated eye mask 100, showing an inside of the mask 100 that would be in contact with the eye region of the user. As shown, the heated eye mask 100 includes a mask body 110 and an adjustable strap 130, with the electrical cord 202 extending from the mask body 110. The mask body 110 is made up of a first layer of surface material 112 configured to be positioned away from the eye region of the user, and a second layer of surface material 114 configured to be in contact with the eye region of the user. The first layer 112 and second layer 114 of surface material are stitched, or otherwise attached, together along an outer perimeter to form the mask body 110. The mask body 110 is also coupled to an adjustable strap 130. In some embodiments, the adjustable strap 130 is elastic. In some embodiments, the adjustable strap 130 is adjustable by way of one or more length adjustment mechanisms 132.

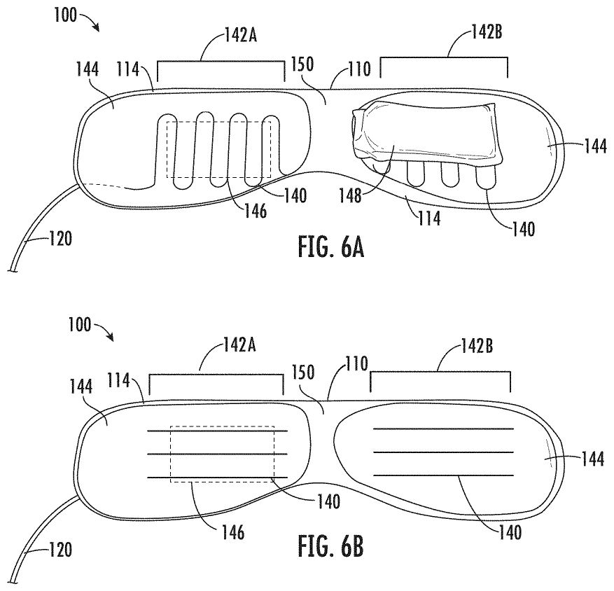

FIGS. 6A-6B show an arrangement of internal elements of the mask body 110 with the first layer of surface material 112 (the outer facing layer) removed. As shown, the mask body 110 includes a heating element 140 positioned between the first layer of surface material 112 and the second layer of surface material 114. In the embodiments shown, the heating element 140 is a flexible fiber, such as a wire. The wire may be a metal fiber heating wire. The wire may be made of nickel-chromium (nichrome). In the embodiment shown in FIG. 6A, the heating element 140 is arranged in a sinusoidal shape. It is to be understood, however, that the heating element can be arranged in any shape or form in order to provide heat to a target area of the mask body 110, such as any number of horizontal lines, such as the three horizontal lines shown in FIG. 6B, any number of vertical lines, a zigzag pattern, etc. The heating element 140 is positioned in a therapeutic region on both sides (right and left eye) of the mask body 110. The therapeutic region includes two therapeutic zones 142A, 142B. A first therapeutic zone 142A is aligned with the Meibomian glands of the right eye and a second therapeutic zone 142B is aligned with the Meibomian glands of the left eye, with a gap between the two therapeutic zones 142A, 142B. In this way, heat is targeted to a certain area over each eye, particularly, the area along the eyelids where the Meibomian glands are found. This targeted therapy that is achieved by positioning the heating element 140 specifically in the therapeutic region provides a more effective treatment for blockage of the Meibomian glands than widespread heat distribution across the entire eye region.

In some embodiments, the heating element 140 is stitched to one or more intermediate layers 144 that are positioned or attached (e.g., by stitching or adhesive) between the first layer of surface material 112 and the second layer of surface material 114. In such embodiments, the heating element 140 may be positioned and stitched in between two intermediate layers 144. The intermediate layer(s) 144 assist in maintaining the heating element 140 in its desired shape and positioned in the therapeutic region 142. The material of the intermediate layer(s) 144 is any material having adequate strength and structure to hold the heating element 140 in position. In some embodiments, there is a piece of thermally conductive material 146 positioned between second layer of surface material 114 and the heating element 140 (i.e., towards the user's eye) in both therapeutic regions 142A, 142B. The thermally conductive material 146 is configured to evenly disperse the heat generated by the heating element 140. The material is preferably a conductive fabric made from, coated, or blended with conductive metals. In some embodiments, a base material, such as cotton, wool, polyester, or nylon, is coated or blended with the conductive metal. The conductive metal may be gold, carbon, titanium, nickel, silver, or copper, for example. The thermal conductive material 146 is preferably a small piece of material that is sized and configured to cover only the therapeutic region 142, therefore further assisting in the targeted therapy to only the area of the eye in which the Meibomian glands are found. To this end, in some embodiments, an additional blocking material 150, for example, a thermal blocking material, may be added in the area of the bridge of the user's nose which prevent the spread of heat between the therapeutic regions 142A, 142B over the right and left eyes.

Still referring to FIG. 6A, in some embodiments, a pillow 148 is provided between the first layer of surface material 112 and the heating element 140 on both sides of mask body 110 (i.e., over each eye). The pillow 148 is configured to apply additional pressure to the heating element 140 to urge the heating element 140 towards the eye socket of the user. Thus, providing an additional therapeutic benefit of maintaining heat and contact throughout the therapy session. In some embodiments, the pillow 148 is made of a polyester material. In some embodiments, the mask body 110 is filled with a flexible, filler material (not shown) to soften the mask body 110 and allow flexibility of the mask to form to the user's eye region. In some embodiments, the filler material is a non-synthetic material which will not heat up when exposed to the heating element 140 (or only minimally) and will also not emit any chemicals or other harmful elements when exposed to heat. For example, the filler material may be flax seed. In addition to being non-synthetic, flax seed also comprises very small seed elements which easily contour to the user's eye region for a comfortable fit. Other types of filler material may be used, such as other materials containing small elements (i.e., beads or seeds) or a soft material (i.e., cotton, polyester, feathers, etc.)

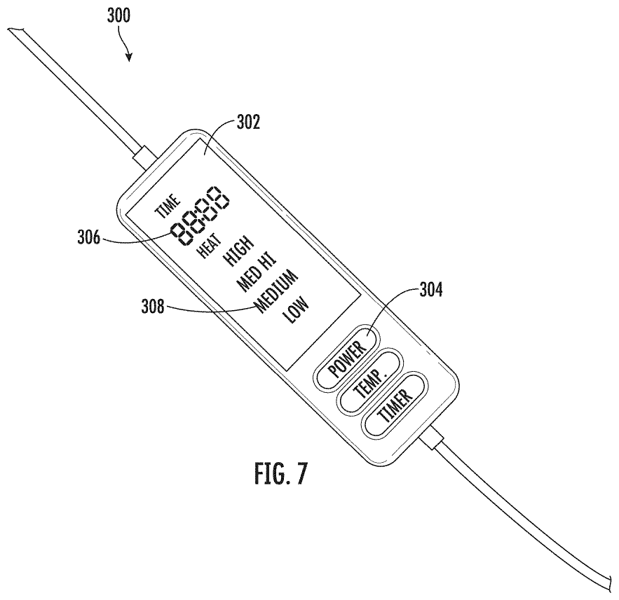

FIG. 7 depicts a controller 300 for controlling the time and temperature settings of the therapy session. In the embodiment shown, the controller 300 includes a display portion including a display 302 and an input portion including one or more buttons, switches, or other type of input mechanism 304. In some embodiments, the display is a touch screen display, and in such cases the display 302 and the input mechanism 304 are a unitary element. In the embodiment shown, the display 302 provides a digital output 306 of the time of the therapy session (it may show time remaining or time elapsed) and an indicator 308 of the temperature setting. The time of the therapy session may be a specific length of time selected by the user, or the controller may allow for selection of one of a plurality of pre-set lengths of time. For example, in some embodiments, the preset lengths of time may start at minimum time (for example, at 10 minutes, 20 minutes, etc.) and increase in two minute intervals. In other embodiments, there may be fewer preset options such as 10 minutes, 15 minutes, 20 minutes, 25 minutes, and 30 minutes. For best results, it is recommended that a user wear the mask twice a day for at least 8 minutes.

In the embodiment shown in FIG. 7, the indicator 308 of the temperature setting is lighting element which illuminates one of four preset temperature settings: low (125)—every second (every other second), medium (135)—2 seconds, medium high (140)—cuts every 3 seconds, or high (145)—heat on continuously. In some embodiments, the low setting provides a temperature of approximately 125 degrees Fahrenheit, the medium setting provides a temperature of approximately 135 degrees Fahrenheit, the medium high setting provides a temperature of approximately 140 degrees Fahrenheit, and the high setting provides a temperature of approximately 145 degrees Fahrenheit. The preset temperatures are achieved by regulating the power supplied to the heating element. For example, to achieve the high temperature, the power is maintained to the heating element 140 throughout the therapy session. To achieve the medium high, medium, and low temperatures, the current is removed every three seconds (three seconds on, one second off), two seconds (two seconds on, one second off), or every other second (one second on, one second off), respectively.

In some embodiments, there may be fewer or more preset temperature settings, or a user may be able to select a specific temperature in degrees for the therapy session. In a preferred embodiment, the temperature settings available to the user are in the range of 120 degrees Fahrenheit to 145 degrees Fahrenheit. The temperature settings may also be shown on the display in a digital format.

The input mechanism(s) 304 on the controller 300 allow a user to select the time and/or temperature settings of the therapy session. In the embodiment shown in FIG. 7, there is a power button, a temperature button, and a timer button. In other embodiments, there may be fewer or more input mechanism(s) such as additional “+” and a “−” buttons to allow a user to increase or decrease the time or temperature. Or, in another example, there may be a single button and a user toggles through an array of menu options in order to select the settings for the therapy session. In yet another example, there are two input mechanisms 304, one for the time setting and the other for the temperature setting.

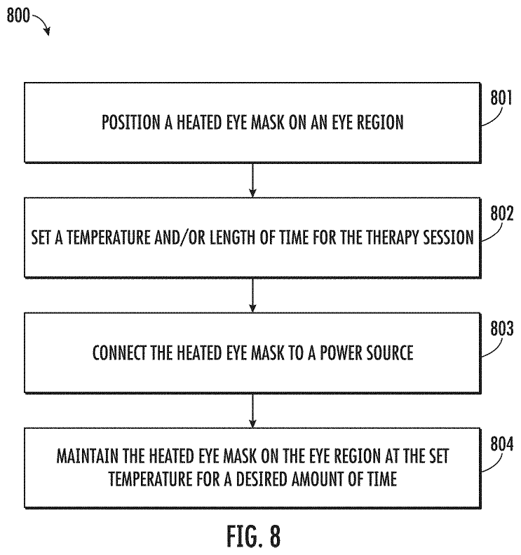

FIG. 8 depicts a method 800 for using the heated eye mask system 10 for treating clogged glands of the eyes. In step 801, a user positions the heated eye mask 100 on the eye region. As described above, the design of the eye mask 100 provides for targeted therapy directly to the area of the eye in which the clogged glands may be found, for example, by the heating element 140 positioned particularly in the therapeutic region 142 and the pillow 148 to bias the heating element 140 towards the eye socket of the user. The user may position and secure the heated eye mask 100 on the eye region by placing the adjustable strap 130 over his or her head and adjusting the strap 130 for a secure fit. In step 802, which may be performed before or after positioning the eye mask 100 on the eye region, the user selects the therapy session parameters, such as the length of time and temperature settings for the therapy session. In step 803, the heated eye mask 100 is connected to a power source 200, such as a standard wall outlet with a 5V plug adapter or to a battery pack. In step 804, the user maintains the heated eye mask 100 on the eye region for the desired length of time and temperature. The user may adjust the temperature or the time, as needed, during the therapy session. As mentioned above, for best results, it is recommended that a user wear the mask twice a day for at least 8 minutes.

What is claimed is:

1. A heated eye mask for treating clogged glands of an eye, the eye mask comprising:
a mask body, the mask body comprising:
a first layer of surface material configured to be positioned away from an eye region of a user and a second layer of surface material configured to contact the eye region of the user;
a heating element disposed between the first and second layers of surface material in a therapeutic region of the mask body; and
a thermally conductive material in contact with the heating element and disposed between the heating element and the second layer of surface material to evenly distribute heat across the therapeutic region of the mask body;
an electrical cord configured to be coupled with a power source and in communication with the heating element; and
an adjustable strap to maintain contact of the mask body with the eye region of the user.

2. The heated eye mask of claim 1, wherein the therapeutic region of the mask body is a portion of the mask body that covers only the area of the eye region of the user extending along the Meibomian glands of the eye.

3. The heated eye mask of claim 1, wherein the heating element is a wire.

4. The heated eye mask of claim 3, wherein the heating element is arranged in a sinusoidal shape.

5. The heated eye mask of claim 1, wherein the heating element is stitched to an intermediate layer of material between the first and second layers of surface material.

6. The heated eye mask of claim 1, wherein the mask body further comprises a pillow positioned in the therapeutic region between the first layer of surface material and the heating element to maintain contact between second layer of surface material and the eye region of the user.

7. The heated eye mask of claim 1, wherein the mask body is filled with flax seed between the first and second layers of surface material.

8. The heated eye mask of claim 1, wherein the power connector is power cord that comprises a USB interface for plugging into a power source.

9. The heated eye mask of claim 1, further comprising a controller coupled to the power source for controlling the heating element.

10. The heated eye mask of claim 1, wherein the adjustable strap is elastic.

11. The heated eye mask of claim 1, wherein the first and second layers of surface material are made up of at least one of: cotton, velvet, silk, polyester, and nylon.

12. A system for treating clogged glands of an eye, comprising:
a heated eye mask;
an electrical cord coupled to the heated eye mask for providing power to the heated eye mask; and
a controller provided on the electrical cord for controlling the heated eye mask.

13. The system of claim 12, wherein the electrical cord comprises a USB interface for connecting with a power supply.

14. The system of 13, wherein the power supply is a USB adapter for plugging into a wall power socket.

15. The system of claim 13, wherein the power supply is a battery pack.

16. The system of claim 12, wherein the controller comprises a timer.

17. The system of claim 16, wherein the timer is configured to control the heated eye mask to provide heat for a specified period of time, wherein the specified period of time is adjustable.

18. The system of claim 17, wherein the specified period of time is at least 8 minutes.

19. The system of claim 12, wherein the controller comprises a temperature control to control the heated eye mask to heat to a specified temperature.

20. The system of claim 19, wherein the specified temperature is in the range of 120 degrees Fahrenheit to 145 degrees Fahrenheit.

21. The system of claim 19, wherein the temperature control provides at least four pre-set temperature levels.

22. A method for treating clogged glands of an eye, comprising:
positioning a heated eye mask on an eye region;
setting at least one of a therapy temperature and a therapy time of use using a controller coupled to the heated eye mask;
providing power to the heated eye mask by connecting the heated eye mask to a power supply; and
maintaining the heated eye mask on the eye region at the set temperature and for a desired amount of time.

23. The method of claim 22, wherein the desired amount of time is at least 8 minutes.

24. The method of claim 22, further comprising repeating the method at least two times a day.
